(19) United States
(12) Patent Application Publication
(10) Pub. No.: US 2009/0163365 A1
(43) Pub. Date: Jun. 25, 2009

(54) Water-swellable hybrid material with inorganic additives and method of Producing same

(76) Inventors: Wulf Bentlage, Frankfurt am Main (DE); Reinmar Peppmoller, Krefeld (DE); Jürgen Kunstmann, Bad Soden (DE); Oliver Zindel, Dreieich (DE)

Correspondence Address: KENYON & KENYON LLP, ONE BROADWAY, NEW YORK, NY, 10004, US

(21) Appl. No.: 11/920,019

(22) PCT Filed: Apr. 4, 2006
(86) PCT No.: PCT/EP2006/003053

§ 371 Date: Feb. 19, 2009

(30) Foreign Application Priority Data
May 7, 2005 (DE) 10 2005 021 221.2

(51) Int. Cl. A01N 25/10; C08F 290/00; C08F 8/00; C08K 3/34; C08K 3/22; C08K 3/32; C08K 3/28; C08K 3/38; C08K 5/21; C08K 5/3437; C08K 5/05; C08L 3/00; C08L 1/00; C08L 5/00; C08L 89/06; A61K 47/30; C12N 1/00; C08F 20/06; C08F 20/04; C08F 28/02; C08F 30/02; C08F 20/56; C08F 20/54; C08F 26/08; C08F 20/10; A01P 3/00; A01P 15/00; A01P 13/00; C09K 3/00; C05F 11/00

(52) U.S. Cl. 504/360; 525/50; 525/55; 524/442; 524/401; 524/417; 524/429; 524/405; 524/211; 524/91; 524/379; 524/47; 524/35; 524/27; 524/13; 524/11; 514/772.1; 435/243; 525/329.7; 525/329.5; 525/328.5; 525/326.6; 525/329.4; 525/328.2; 525/326.7; 525/330.3; 525/374; 525/386; 525/384; 252/194; 71/27

(57) ABSTRACT

The present invention relates to a water-swellable material comprising an inherently crosslinked polymer matrix and inorganic solid particles bound therein with a time-dependent swelling behavior that corresponds to a water uptake of at least 7.5 times the inherent weight of the hybrid material within one hour, as well as the applications thereof. The present invention further relates to a method for manufacture of such a water-swellable hybrid material.

The present invention relates to a novel water-swellable hybrid material comprising an inherently crosslinked polymer matrix and inorganic solid particles bound therein with a time-dependent swelling behavior that corresponds to a water uptake of at least 7.5 times the inherent weight of the hybrid material within one hour as well as the applications thereof. The present invention also relates to a method for producing a water-swellable hybrid material, which consists of providing a reaction mixture including at least one polymerizable component and at least one suitable solvent, where the pH of the reaction mixture is less than 7; blending inorganic solid particles and at least one crosslinking agent into the reaction mixture; initiating and controlling the polymerization reaction so that a spongy, water-swellable hybrid material comprising an inherently crosslinked polymer matrix and inorganic solid particles bound therein is obtained with a volume increase in relation to the volume of the reaction mixture.

### BACKGROUND OF THE INVENTION

Acrylate (co)polymers that take up water or aqueous liquids to form hydrogels have already been described. These are usually prepared by the methods of inverse suspension polymerization or emulsion polymerization, as described in U.S. Pat. No. 4,286,082, German Patent DE 27 06 135, U.S. Pat. No. 4,340,706 and German Patent DE 28 40 010. Polymer products obtained in this way are also known as super absorbents and are generally used in the fields of personal hygiene and sanitation. However, there have also been proposals for using the hydrogel-forming polymer products produced for the personal hygiene sector as water storage devices in the botanical sector, e.g., as described in German Patent Application DE 101 14 169.6 or also in International Patent Application WO 03/000621.

In the case of materials such as those described in International Patent WO 03/000621, it has been found that superabsorbents containing eruptive substances obey their own laws in both production and use because of their polyvalent metal ion content, where the metal ions can act as chelating agents. In particular it has been found that the production process as well as the powdered minerals used have a significant influence on the swelling behavior of the products described in this international patent application. For example, it has been found that particles that require a relatively long period of time to swell completely, in some cases 24 hours or more, are obtained when these conventional materials are produced from a basic polymerization mixture.

### ABSTRACT OF THE INVENTION

An object of the present invention is therefore to provide a product that no longer requires such a long swelling time.

Furthermore, an object is to make available a water-swellable hybrid material which will provide the mineral and nutrient supply required for a plant, for example, in the form of a crosslinked polymer matrix containing ballast, so that the water storage capacity and/or swellability of the hybrid material is not impaired.

In addition, another object is to make available methods for producing hybrid materials containing minerals and inorganic solids for a variety of applications, leading to products that are essentially free of monomer residues.

The solutions to the objects of the present invention are provided by the subject-matter of the independent product claims, process claims and use claims. Advantageous embodiments are provided in the respective subclaims.

### DESCRIPTION OF THE FIGURES

FIG. 1 shows the spongy structure of an exemplary hybrid material according to claim 1 of the present invention, with FIG. 1A showing the dry material with a needle for size comparison and FIG. 1B showing the same material in a swollen, water-saturated state.

FIG. 2 shows the swelling behavior of the material according to Example 4 (bottom curve, triangles) in comparison with the hybrid material according to Example 1 (upper curve, squares).

FIG. 3 shows the different heights of growth of grass in a comparison of plant substrate without the addition of the inventive hybrid material (pots on the left) with plant substrate containing the hybrid material (pots on the right) watered with 57 mL every three days, and FIG. 3B shows an enlarged detail of the photograph from FIG. 3A.

FIG. 4 shows the different heights of growth of grass in a comparison of plant substrate without the addition of the inventive hybrid material (pots on the left) with plant substrate containing the hybrid material (pots on the right), watered with 57 mL every six days, and FIG. 4B shows an enlarged detail of the photograph from FIG. 4A.

### DETAILED DESCRIPTION OF EXEMPLARY EMBODIMENTS

To achieve the objects defined above as well as other objects, the present invention provides a novel water-swellable hybrid material comprising an inherently crosslinked polymer matrix and inorganic solid particles bound therein, said hybrid material having extraordinary properties, especially with regard to its swelling behavior. Without being limited to a certain theory, it is presently assumed that the novel properties of the hybrid material may be due to the process by which it is produced.

According to an exemplary embodiment of the present invention, a water-swellable hybrid material and a production process for it are provided, comprising an inherently crosslinked polymer matrix with inorganic solid particles bound therein, whereby the hybrid material swells rapidly on coming in contact with aqueous liquids such as water, taking up water in the process, and reaching its maximum uptake capacity at the earliest possible point in time. The term “water-swellable” in the present context is understood to refer to a material that undergoes an increase in its natural volume, but preferably does not alter its chemical structure, on coming in contact with water or aqueous liquids such as salt solutions, body fluids, etc. or other protic polar solvents, including the uptake of these liquids. The term “inherently crosslinked polymer matrix” as used here refers to a three-dimensionally crosslinked homopolymer or copolymer having an open and/or closed pore structure, containing the inorganic solid particles preferably in a bound form, e.g., being chemically bound and/or occluded within the pore structure. It is preferable for the inherently crosslinked polymer matrix and/or the hybrid material to essentially retain its structure even in the water-saturated state. The inherently crosslinked polymer matrix and/or the hybrid material may preferably take up water to the saturation limit without forming a hydrogel, i.e., the polymer matrix and/or the hybrid material does not form a liquid hydrogel with uptake of water, as is usually the case with superabsorbents. Unless otherwise stated explicitly, amounts given in weight percent are based on the total weight of the dry hybrid material, i.e., at a water content of approximately <0.1 wt %, for example, and/or after 12 hours of drying of the material, preferably at approximately 40° C., preferably in a forced-air circulation oven. All numerical values and ranges given here as well as property data and parameters are to be understood as essentially combinable in any form, unless explicitly stated otherwise.

The swelling behavior of the hybrid material can be determined, for example, by bringing the hybrid material in contact with a sufficient amount of deionized water, for example, typically at room temperature of approximately 20-23° C., preferably 20° C., and by weighing the dripped off material at certain intervals of time.

According to an exemplary embodiment of the present invention, the hybrid material has a time-dependent swelling behavior which corresponds to a water uptake of at least 7.5 times the inherent weight of the dry hybrid material within one hour, preferably at least ten times, especially preferably at least 12.5 times and most preferably at least 15 times the inherent weight of the dry hybrid material within the first hour. After two hours, water uptake by the hybrid material may amount to at least ten times the inherent weight of the dry hybrid material, preferably at least 12.5 times, especially preferably at least 15 times and most preferably at least 17.5 times the inherent weight of the dry hybrid material. After three hours, water uptake by the hybrid material may amount to at least 12.5 times the inherent weight of the dry hybrid material, preferably at least 15 times, especially preferably 17.5 times, most preferably at least 20 times the inherent weight of the dry hybrid material. Water uptake by the hybrid material after 24 hours can amount to at least 15 times, preferably 20 times, especially preferably at least 25 times, most preferably at least 30 times the inherent weight of the dry hybrid material and may even amount to more than 50 times the inherent weight of the dry hybrid material, without forming a hydrogel like that associated with the conventional superabsorbent materials.

The solids containing water-swellable hybrid material according to the present invention differs from conventional materials in its production and composition. It has a high swellability in particular, and in the undried state containing residual moisture, it is directly comparable to humus, for example. A suction effect may occur in the swelling process in aqueous liquids owing to the increase in pore volume, possibly resulting in an uptake of liquid which goes beyond the absorption capacity of the polymer matrix.

In exemplary embodiments of the present invention, the hybrid material is essentially free of alkali silicate and/or essentially free of monomer residues. According to this invention, the term “essentially free of monomer residues” is understood to refer to a material containing less than 1000 ppm, preferably less than 500 ppm and especially preferably less than 300 ppm, optionally even less than 100 ppm or less than 50 ppm monomer residues.

In certain exemplary embodiments, the polymer matrix includes at least one homopolymer and/or copolymer of ethylenically unsaturated components, in particular acrylic acid or acrylic acid derivatives. The polymer matrix may be formed by polymerization of at least one water-soluble, ethylenically unsaturated monomer containing acid groups and optionally in addition at least one water-soluble ethylenically unsaturated comonomer that can be polymerized therewith; at least one crosslinking agent and optionally additional water-soluble polymer may be added, preferably in amounts of approximately 0.01 to 5 wt %, typically 0.1 to 2 wt %. Examples of crosslinking agents that may be used include substances containing at least two ethylenically unsaturated groups or at least one ethylenically unsaturated group and at least one other functional group which his reactive with respect to acid groups. Suitable monomers, comonomers, water-soluble polymers, crosslinking agents and other polymer constituents are described further below in conjunction with the production process.

In certain embodiments, the monomers and/or comonomers may optionally be partially neutralized with basic substances such as sodium hydroxide, ammonia solution, ammonium hydroxide, potassium hydroxide, sodium carbonate, potassium carbonate, guanidine and guanidine carbonate or by using alkaline powdered rock/minerals as inorganic solid particles.

In exemplary embodiments of the present invention, the weight ratio of polymer matrix to the inorganic solid particles can be between 99:1 and 1:99, preferably between about 90:10 and 10:90, or optionally between about 70:30 and about 30:70. In preferred exemplary embodiments, the amount of inorganic solids is at least about 50 wt %, preferably at least about 60 wt % and especially preferably at least about 70 wt % or even at least 80 wt %. The polymer content may be at least about 5 wt %, preferably at least about 10 wt % or at least about 20 wt %.

The inorganic solid particles may include, for example, ground minerals, slags or powdered rocks including at least one mineral selected from the group comprising of quartz sand, clay, shale, sedimentary rocks, meteorite rocks, eruptive rocks such as powdered lava rock, graywacke, gneiss, trass, basalt, diabase, dolomite, magnesite, bentonite, pyrogenic silica and feldspar. These solid particles, bound into the inherently crosslinked polymer matrix of the hybrid material, can greatly improve the soil structure and soil climate in agricultural and/or botanical applications, for example, and through the addition of fertilizers from the group of conventional K, N, P fertilizers and/or trace elements such as iron, zinc, etc. may constitute an optimum nutrient source for plants, fulfilling all the important conditions for their growth. Due to the porous spongy structure of the hybrid material of the present invention, the soil capillarity can be improved while at the same time the properties of the soil are influenced in a positive sense due to the presence of finely ground minerals, especially fine sand. Furthermore, the mineral content of the hybrid materials makes the product heavier so it is prevented from floating when soil wetness is high, for example.

Since the inorganic ingredients of the inventive hybrid material can influence the polymerization process and thus the sponge structure of the hybrid material especially with regard to the trace elements and/or in conjunction with the particle size, so it has proven advantageous in certain exemplary embodiments of this invention to select a suitable particle size of the inorganic solid particles. At the same time, this powdered rock mineral constitutes a source of mineral nutrients for plants, so the degree of milling can be selected so that the particle sizes of the inorganic solid particles are less than 200 μm, preferably less than 100 μm.

In certain embodiments of the present invention, the hybrid material may include, for example, clay materials such as bentonite, montmorillonite, phyllosilicates, zeolites, etc. These clay minerals may have the ability to take up even small amounts of liquids and to bind cations, for example. They may therefore contribute toward the strength and swelling behavior of the hybrid material. Their particle sizes may especially preferably be between about 0.1 mm and 8 mm, preferably between about 0.3 mm and 5 mm. The amount ratio in certain exemplary embodiments of the hybrid material of the present invention may be between approximately 5 wt % and 60 wt %, based on the total weight of the hybrid material in the dry state.

The other inorganic solids that are preferably added to the inventive hybrid material mainly also have the effect of making the product heavier and may thus fulfill an important function. The inventive hybrid materials may additionally contain other solid organic or inorganic additives, optionally finely ground, in subordinate amounts.

Furthermore, the hybrid material may optionally contain inorganic additives that are soluble in water and/or dissolved in water, consisting of at least one additive selected from alkali silicate, potassium water glass, sodium water glass, alkali hydroxide, potassium hydroxide, sodium hydroxide, silica, alkali phosphate, alkali nitrate, alkaline earth hydrogen phosphate, phosphoric acid, magnesium oxide, magnesium hydroxide, magnesium carbonate, iron oxides, iron salts, especially Fe(II) salts and/or boric acid.

The properties of the inventive hybrid material can be further modified and/or improved if it additionally contains water-soluble or water-insoluble organic additives or solid, optionally finely pulverized or dissolved in water, e.g., urea, uric acid, e.g., for evolution of CO₂ during polymerization and/or as a fertilizing nitrogen source, e.g., as a fertilizer, glycol, glycerol, polyethylene glycol, polysaccharides, starch, starch derivatives, cellulose, wood, straw, peat, recycled paper, chromium-free leather and recycled wood or recycled plastic granules or plastic granules, fibers or nonwovens, e.g., for modification of physical properties, depending on the intended application.

In certain embodiments, the inventive hybrid materials may contain microorganisms such as algae, bacteria, yeasts, fungi, fungal spores or the like, e.g., as a supply of nutrients. Coloring agents and/or flavoring agents may also be added to improve the sensory properties, if desired. Fungicides, pesticides, herbicides and the like may also be added, if desired, to achieve an environmentally friendly non-aerosol means of applying the active ingredients near the plant roots, optionally with a depot action and/or with a slow release, optionally a controlled release.

After being prepared in an aqueous medium, the hybrid material may have a residual moisture contest of at least about 0.1 wt %, based on the total weight of the residually moist material, preferably up to about 60 wt %, especially preferably about 20 wt % to 40 wt %, especially about 35 wt % at 20° C. By partial drying, the residual moisture content can be adjusted to meet the desired requirements.

Due to its spongy structure arising from its production process, the inventive hybrid material according to certain exemplary embodiments has advantageous mechanical properties for a variety of applications. In one exemplary embodiment, the hybrid material may have a Shore A hardness (according to DIN 53505) of at least about 25, preferably about 30 to 50, after one hour of air drying at 40° C. In the wet-from-production condition immediately after production, with a moisture content of about 30 wt % to 40 wt %, the hybrid material may additionally or alternatively have a Shore A hardness (DIN 53505) of at least about 15, preferably at least about 20 to 30. Furthermore, when saturated after storing the material for 24 hours in deionized water, the hybrid material may additionally or alternatively have a Shore A hardness (DIN 53505) of at least about 1, preferably about 2 to 10.

The specific gravity of the hybrid material is at least 1 g/cm³, preferably between about 1.1 and 5 g/cm³, preferably between about 1.2 and 2.5 g/cm³, depending on the solid particles used and/or the polymer ingredients.

### Production Process

According to the conventional production process described in WO 03/000621, the starting materials are minerals in the form of an aqueous slurry containing alkali carbonate and/or carbon dioxide at a neutral or alkaline pH, and the ethylenically unsaturated monomers containing acid groups and including the crosslinking agent are then added, whereupon carbon dioxide is released, resulting in foaming. Polymerization is performed after the foaming stops. As an alternative to this, at a neutral or alkaline pH, the minerals in the form of an aqueous starting slurry may be used as the starting materials together with the alkali substances for partial neutralization of the acid groups of the monomers and polymerization is then performed.

In this way, neutral or weakly alkaline products having a stable sponge structure which absorbs large amounts of water in the neutral pH state, much like the superabsorbents, are usually obtained. With both conventional methods, the minerals are always used as the starting materials and the monomers are only added subsequently.

It has surprisingly now been found that by modifying the sequence of addition of the reactants and optionally also selecting suitable pH ranges in the reaction mixture, through suitable control of the polymerization reaction, it is possible to greatly improve the properties of the hybrid material and in particular the swelling behavior. It has also been found that by suitable control of the polymerization conditions, it is possible to largely omit the addition of carbonates and similar compounds to release the gas for foaming the hybrid material to produce its sponge structure.

It has been found that starting with the monomers containing the acid groups and then adding the minerals in this order may be especially advantageous for the development of an essentially homogeneous sponge structure in the resulting material. The polymerization proceeds more uniformly than with the conventional processes and yields products having a definitely improved initial swelling, i.e., the hybrid materials produced by this method swell up very rapidly after adding water, reaching their maximum water uptake at an early point in time.

The polymerization reactions of ethylenically unsaturated monomers containing acid groups are typically exothermic, which is why the reaction is initiated at the lowest possible temperature (typically around 0° C.) in the conventional superabsorbent production processes, and the heat of reaction is subsequently removed continuously to keep the temperature as low as possible.

In an exemplary embodiment of the present invention, it has been found that by suitable control of the polymerization reaction, at least partial vaporization of the solvent can be achieved, so that a spongy, water-swollen hybrid material including an inherently crosslinked polymer matrix with inorganic solid particles bound therein is obtained with an increase in volume in relation to the volume of the reaction mixture. This hybrid material has an excellent swelling behavior in particular, especially having a much more rapid initial water uptake with excellent mechanical stability in the saturated state.

Furthermore, it has been found that by starting with the acidic monomers at a pH of less than 7 and then adding the inorganic solid particles, an improved binding of minerals in the spongy polymer matrix can also be achieved without having any negative effect on the swelling performance. This is possible even if the inorganic solid particles such as eruptive rocks have a high trace element or electrolyte content which in conventional methods would typically lead to a delay in polymerization and would result in a different material structure which would usually have a slow initial swelling behavior.

In an exemplary embodiment of the present invention, a process for producing a water-swellable hybrid material comprising an inherently crosslinked polymer matrix with inorganic solid particles bound therein is made available, comprising the following steps:
a) providing a reaction mixture comprising at least one polymerizable component and at least one suitable solvent, the pH of the reaction mixture being less than 7;
b) then mixing inorganic solid particles into the reaction mixture;
c) adding at least one crosslinking agent;
d) initiating the polymerization reaction; and
e) controlling the polymerization reaction so that a spongy, water-swellable hybrid material comprising an inherently crosslinked polymer matrix with inorganic solid particles bound therein is obtained, accompanied by an increase in volume in relation to the volume of the reaction mixture.

As already mentioned, the polymer matrix may be composed of crosslinked homopolymers and/or copolymers based on ethylenically unsaturated polymers containing acid groups, e.g., polyacrylates. In a preferred exemplary embodiment of the present invention, a process for producing a water-swellable hybrid material comprising an inherently crosslinked polymer matrix and inorganic solid particles bound therein is therefore made available, comprising the following steps:
a) providing a reaction mixture comprising at least one ethylenically unsaturated monomer containing acid groups and at least one suitable solvent, the pH of the reaction mixture being less than 7;
b) then mixing inorganic solid particles into the reaction mixture;
c) adding at least one crosslinking agent;
d) initiating the polymerization reaction; and
e) controlling the polymerization reaction so that a spongy, water-swellable hybrid material comprising an inherently crosslinked polymer matrix and inorganic solid particles contained therein is obtained, accompanied by an increase in volume in relation to the volume of the reaction mixture.

The at least one polymerizable component may be selected from water-soluble ethylenically unsaturated monomers containing acid groups, comprising at least one selected from the group consisting of acrylic acid, methacrylic acid, ethacrylic acid, sorbic acid, maleic acid, fumaric acid, itaconic acid, vinylsulfonic acid, methacrylamino-alkylsulfonic acid, vinylphosphonic acid or vinylbenzene-phosphonic acid.

The amount of comonomers in the reaction mixture may be 0 to 50 wt %, based on the polymerizable components of the monomer reaction mixture. Water-soluble ethylenically unsaturated comonomers may be selected from at least one consisting of unsaturated amines such as acrylamide, methacrylamide, N-alkylacrylamide, N-alkylmethacrylamide, N-dialkylaminoacrylamide, N-dialkylaminomethacrylamide, N-methylolacrylamide, N-methylolmethacrylamide, N-vinylamide, N-vinylformamide, N-vinylacetamide, N-vinyl-n-methyl-acetamide, N-vinyl-n-methylacetamide, N-vinyl-n-formamide, vinylpyrrolidone, hydroxyethylene acrylate, hydroxyethyl methacrylate, acrylate esters and/or methacrylate esters. Acrylic acid is especially preferred as a monomer, preferably without the addition of comonomers.

Water-soluble polymers may also be added to the monomer reaction mixture in amounts of up to 30 wt %, based on the polymerizable substance of the monomer reaction mixture. Examples of soluble polymers that may be used include homopolymers or copolymers of the aforementioned monomers or comonomers, partially saponified polyvinyl acetate, polyvinyl alcohol, starch, starch derivatives, graft-polymerized starch, cellulose and cellulose derivatives such as carboxymethyl cellulose, hydroxymethyl cellulose and galactomannose as well as its alkoxylated derivatives plus any desired mixtures of these. These water-soluble polymers are essentially bound physically.

The monomers and/or comonomers are used as the starting material in at least one suitable solvent. In an exemplary embodiment of the invention, the at least one solvent may include protic polar solvents such as water, aqueous solutions, alcohols such as methanol, ethanol; alkylamines, tetrahydrofuran, dioxane and any mixtures thereof, but especially preferably water. Furthermore, these protic polar solvents may optionally also be used in mixtures with aprotic and/or apolar solvents, optionally with the addition of surfactants, emulsifiers or other amphiphilic substances in order to obtain the most homogeneous possible reaction mixture.

In preferred exemplary embodiments of the present invention, the pH of the reaction mixture may be less than 7 prior to addition of the inorganic solid particles. The pH is especially preferably less than 6.8, preferably less than 6.5, especially less than 6 or less than 5, e.g., between pH 0 and pH 6 or between pH 1 and pH 5.

At least one crosslinking agent may be added to the reaction mixture of solvent and at least one polymerizable component. Preferably the at least one crosslinking agent is added in an amount of 0.01 wt % to 5 wt %, preferably 0.1 wt % to 2.0 wt % based on the total amount of polymerizable monomers. All substances containing at least two ethylenically unsaturated groups or at least one ethylenically unsaturated group and at least one other functional group that is reactive with acid groups may be used as the crosslinking agent. Examples of representatives that can be mentioned here include methylenebisacrylamide, mono-, di- and polyesters of acrylic acid, methacrylic acid, itaconic acid and maleic acid of polyvalent alcohols such as butanediol, hexanediol, polyethylene glycol, trimethylolpropane, pentaerythritol, glycerol and polyglycerol as well as the alkoxylated homologs resulting therefrom, e.g., butanediol diacrylate as well as the esters of these acids with allyl alcohol and its alkoxylated homologs. Other examples include N-diallyl-acrylamide, diallyl phthalate, triallyl citrate, tri-monoallyl-polyethylene glycol ether citrate, allylacryl-amide, triallyl citrate, trimonoallyl, polyethylene glycol ether citrate as well as the allyl ethers of diols and polyols and their ethoxylates representatives of the species mentioned last include polyallyl ethers of glycerol, trimethylol propane, pentaerythritol and the ethoxylates thereof as well as tetra-allyloxyethane and polyglycidylallyl ethers such as ethylene glycol diglycidyl ether and glycerol glycidyl ether. Other suitable examples include diamines and their salts with at least two ethylenically unsaturated substituents, such as diamine and triallylamine and tetra-allylammonium chloride. In exemplary embodiments of the present invention, optionally at least two different crosslinking agents, preferably differing in their hydrolysis stability, or at least three crosslinking agents may be used. Preferred crosslinking agents in the case of the at least two crosslinking agents include butanediol diacrylate and methylenebisacrylamide.

The inorganic solid particles may be added before, after or together with the at least one crosslinking agent. The inorganic solid particles are preferably added to the reaction mixture already containing the at least one polymerizable component. By starting with the polymerizable component(s), especially ethylenically unsaturated monomers containing acid groups, and especially at an acidic pH and then subsequently adding the inorganic solid particles, it is possible to produce hybrid materials with an especially pronounced initial swelling behavior, i.e., rapid swelling immediately after coming in contact with water, for example. The inorganic solid particles may include ground minerals, slags or powdered rocks, for example, containing at least one material selected from quartz sand, clay, shale, sedimentary rocks, meteorite rocks, eruptive rocks such as powdered lava rocks, graywacke, gneiss, trass, basalt, diabase, dolomite, magnesite, bentonite, pyrogenic silica and feldspar. These solid particles may also be selected from fertilizers from the group of conventional K, N, P fertilizers which are added to the reaction mixture, optionally in addition to the minerals listed above.

The amount of inorganic solid particles may be selected and adjusted as needed in accordance with the desired application, but the usual amounts and quantity ratios are given above. Hybrid materials with a high solids content are preferred, preferably those with an inorganic solids content of more than 60 wt %, based on dry hybrid material. The eruptive rock content, e.g., lava rock, is preferably less than 35 wt % based on the dry hybrid material, especially less than 30 wt %, especially preferably less than 25 wt %. The inorganic solid particles especially preferably do not contain any minerals or salts that release carbon dioxide in the presence of acid.

By using basic solid particles in a suitable amount, the at least one polymerizable component may be at least partially hydrolyzed and thus the pH, the course of polymerization and ultimately the product structure may be modified in suitable manner. Preferably approximately max. 80 mol %, e.g., approximately 60 mol % to 80 mol % of the acid groups of the monomers are neutralized and in exemplary embodiments max. 40 mol % of the acid groups of the monomers are neutralized. As an alternative or in addition to the use of basic solid particles, a partial neutralization or adjustment of pH may optionally be performed by adding at least one basic substance, e.g., an alkaline earth hydroxide and/or alkali hydroxide, lime, alkylamines, ammonia water, etc. as well as the compounds mentioned above.

By suitable homogenization measures such as stirring, the solid particles may be essentially uniformly distributed in the reaction mixture, with the stirring preferably also being continued during polymerization.

To initiate free radical polymerization, conventional redox systems may be used, e.g., peroxo or azo compounds such as potassium peroxomonosulfate, potassium peroxodisulfate, tert-butyl hydroperoxide, 2,2′-azobis(2-methylene-propion-amidine) dihydrochloride or hydrogen peroxide, optionally together with one or more reducing agents such as potassium sulfite, potassium disulfite, potassium formamidine sulfonate and ascorbic acid. The oxidizing agent is preferably present in the starting mixture. In especially preferred exemplary embodiments of this polymerization process, the polymerization may also be initiated by photocatalysis in conjunction with suitable sensitizers.

In exemplary embodiments of the present invention, to promote the development of a porous sponge structure of the hybrid material, the polymerization reaction may be controlled in such a way that the hybrid material is formed with an increase in volume in relation to the volume of the reaction mixture. The heat of the reaction in particular is preferably controlled through suitable measures.

In exemplary embodiments of the present invention, the reaction heat of the exothermic polymerization reaction can be controlled so that approximately 0.3 wt % to 30 wt %, preferably approximately 2 wt % to 15 wt % of the at least one solvent is evaporated. The evaporating solvent acts as a foaming gas, causing the hybrid material to foam up and increase in volume, so that typically it is not necessary to add foaming agents such as gas-evolving substances, especially since certain monomers are capable of releasing gases that are optionally split off, e.g., carbon dioxide, even in polymerization. If desired, however, at least one gas-forming agent may also be added, e.g., carbonate salts and/or urea, to at least partially induce or support the increase in volume. In especially preferred exemplary embodiments of the present invention, no carbonate salt and/or no mineral substance and/or no substance in general is added to the reaction solution and/or the hybrid material and in particular no inorganic substance which releases carbon dioxide in the presence of acids. If carbon dioxide is to be released in addition to the evolution of water vapor to support the formation of the sponge structure of the hybrid material, then preferably organic compounds such as urea or the like which represent an advantageous source of nitrogen in addition to releasing carbon dioxide are used for this purpose.

In other exemplary embodiments of the present invention, the heat of reaction may alternatively or additionally also be controlled by the quantity ratio of the at least one polymerizable component to the at least one suitable solvent and/or via the volume of the solvent. The quantity ratio of the at least one polymerizable component to the at least one suitable solvent is preferably between approximately 1:1 and 1:5. Alternatively or additionally, the reaction heat may also be controlled by cooling the reaction mixture.

In exemplary embodiments of the present invention, an increase in volume in relation to the volume of the reaction mixture before the onset of the polymerization reaction of at least 10%, preferably at least 20%, especially at least 50% and especially preferably at least 100% can be induced by controlling the polymerization reaction.

The average reaction temperature of the polymerization reaction is preferably kept between about 50° C. and 130° C., preferably from about 60° C. to 110° C., especially from about 70° C. to 100° C. The starting temperature of the reaction mixture may be between about 4° C. and about 40° C., preferably about 15° C. to about 30° C., e.g., at about room temperature, i.e., about 20° C. to 22° C.

In certain exemplary embodiments of the present invention, organic solid particles as listed above may additionally be incorporated, e.g., in step b), so they can also be found in the polymer matrix. Preferred examples include at least one organic substance from the group of microorganisms, bacteria, fungi, algae, yeasts, fungicides, pesticides, herbicides, cellulose, starch, derivatives of starch, plastics or polysaccharides; wood, straw, peat, recycled paper, chromium-free leather and recycled granules, plastic granules, fibers or nonwovens.

In addition, at least one water-soluble or water-swellable additive and/or an additive dissolved in water, such as those listed above, may also be added to the reaction mixture. Preferred examples include at least one selected from alkali silicate, potassium water glass, sodium water glass, potassium hydroxide, sodium hydroxide or urea.

In contrast to the conventional methods, with the method described herein, an aftertreatment such as post-crosslinking, neutralization and the like is usually not necessary, i.e., the hybrid material is obtained in a form directly suitable for the intended application described herein by the method described herein.

The hybrid material according to the present invention can be obtained essentially free of monomer residues by a suitable choice of components and/or through suitable process control, although that need not always be the case. In particular for applications in the agricultural area, however, it is advantageous that the low residual monomer content still optionally remaining in the product after polymerization precludes any risk to natural life. According to conventional methods in the area of superabsorbents, the polymer products may be subjected to an intense drying after they are produced to remove the monomer residues. The drying temperatures used are typically far above the boiling point of acrylic acid (b.p.: 142° C.), generally above 170° C. These conditions are necessarily also associated with the risk of incipient product decomposition.

According to a certain exemplary embodiment of the present invention, to reduce and/or remove the residual monomer content in the hybrid material, a cleaning method may therefore be employed. According to this method, the hybrid material can be thermally or chemically aftertreated, e.g., by heating the hybrid material in a circulating oven or, especially preferably, with superheated steam at temperatures of about 100° C. to about 150° C., optionally under pressure. This may be accomplished, for example, by adding products having a residual acrylic acid monomer content or other impurities to a thermally insulated pressurized pot with a lower steam feed line, and an upper excess pressure valve and then subjecting them to a steam treatment. The temperature of the steam may advantageously be adjusted between 100° C. and 150° C., especially between 100° C. and 120° C., or correspondingly lower when working under pressure.

It has surprisingly been found that this steam treatment already produced a definite reduction in the acrylic acid content, i.e., the monomer content after only a short period of time. It may be regarded as especially advantageous that ammonium polycarboxylates could also be treated with steam without any risk of decomposing. If the treatment is additionally carried out under pressure, it may be associated with a reduction in the water content in the hybrid material at the same time, so that in this way at least a partial drying can also be performed. In addition, before, during or at the end of the steam process, there is also the possibility of achieving the removal of any remaining minimal quantities of acrylic acid or other monomers or comonomers and/or accelerating it by adding sulfur dioxide gas, for example, or ammonia to the steam or applying it separately.

With this aftertreatment and/or this cleaning method, the residual monomer content of all the products containing polycarboxylates and having an acrylic acid content, i.e., any type of superabsorbent materials, in particular also the hybrid materials such as those described in the present invention may advantageously be reduced in this way to a level that rules out or at least minimizes any risk to natural life, and to do so preferably without total drying.

The aftertreatment steps described here may also be performed in addition or as an alternative to post-crosslinking, partial hydrolysis and/or simply for drying and/or adjusting a defined residual moisture content of the hybrid material. Suitable residual moisture contents are defined above. The hybrid material is preferably not dried completely after its production.

An optional object of the present invention is therefore a method for removing residual acrylic acid from particulate polymer products containing polycarboxylate and mixtures containing these polymer products by treating with steam at a temperature of about 100° C. to 160° C., optionally under pressure. The treatment is preferably performed with steam at a temperature of about 100° C. to 150° C., especially at about 20° C. to 140° C., optionally lower when working under pressure. Optionally ammonia or sulfur dioxide may also be mixed with the steam, preferably in small amounts, e.g., about 0.1 to 10 vol %, e.g., 0.1 to 5 vol % based on the volume of steam.

It has surprisingly also been found that not only polyacrylates or products containing polyacrylates can be freed of residual monomers such as acrylic acid by using steam but also, especially in the case of ester-like chain linkages, the capacity to uptake water is significantly increased again. Without being fixated on a certain theory, this allows the conclusion that a few chain bridges are optionally broken and this effect thus comes about due to the resulting chain lengthening between two crosslinking points. Thus a given water uptake capacity can optionally be increased again subsequently, e.g., by using at least two types of crosslinking agents having differing hydrolysis stability or at least two or more types of crosslinking agent by means of a steam treatment or heating the moist product.

Another optional object of the present invention is therefore a method for increasing the water uptake capacity of polymer products containing polycarboxylate and mixtures thereof by means of a steam treatment as described above or by brief heating (approximately 10 seconds to one hour) at a high temperature (at least 140° C., preferably at least 150° C.) after polymerization in a moist state.

All particulate products containing polycarboxylates, including the ammonium salts thereof, i.e., superabsorbents, as well as the hybrid material having a residual acrylic acid content can be treated by this method to reduce their residual monomer content to a level at which there is no longer any risk or an odor burden and to do so without intense drying. Furthermore, the water uptake capacity of the polymer can be further increased. If a completely anhydrous carboxylate-containing product is desired in the process, a gentle, emission-free open drying may optionally be performed subsequently.

Since the products are usually obtained in the form of blocks or larger crumbs after their production, a size reduction step is usually provided before further use, with conventional shredding or size reduction methods being suitable for optionally elastic spongy hybrid materials. The first step is usually chopping, which results in disks, mats or smaller blocks. If the mat shape is retained, by further cutting or stamping a wide variety of shapes can be obtained. For example, it is possible to produce rectangular rods which subsequently supply the plant roots with the minerals and fertilizer required for growth when they are inserted in their nutrient area. However, a chopping machine may also be used, in which case it is possible to directly produce soil-like crumbs of any adjustable particle size. These may be adapted especially well to humus in terms of both appearance and properties. In the condition fresh from production, the material may still have a certain tackiness which can be utilized to add additional solids and to produce a wide variety of shapes and structures by simply comprising the crumbs.

Size reduction methods in which the energy input is as low as possible are preferred, e.g., slowly rotating cutting/shredding units (shredders) of designs having one or more shafts or the like. The energy input in size reduction or shredding is then selected in a suitable manner, preferably not amounting to more than 100 W/kg, especially no more than 30 W/kg.

The hybrid materials, e.g., in granular or crumb form are excellently suited for use as soil additives in a variety of applications. When incorporated as soil additives in a suitable amount into soil, sand, humus, peat and the like, they promote germination, growth and cultivation of plants due to their water uptake and storage capacity and can therefore yield good plant results even when added to poor soils under poor weather conditions. Meanwhile they also allow a restriction on watering intervals and therefore are especially beneficial in farming areas of low rainfall. An especially preferred application of the inventive products is for admixture to soils in arid regions for storing water.

It is also possible to use the inventive hybrid materials alone for cultivating plants. A special embodiment of this is use of these products in plant containers connected to a water reservoir, e.g., by capillary rods from which the product sponge obtains the water which is then taken up by the plant roots.

The crumb of the inventive product with its pores and pockets is excellently suited as a vehicle for a wide variety of solids. Of the numerous possible combinations, subsequent mixing with castor bean scrap should be mentioned here. Castor bean scrap is obtained in the production of castor oil and is considered to be a solid fertilizer. In alternative embodiments, instead of castor bean scrap, rapeseed scrap, a waste product of canola oil production, may also be used. Mixtures of these and other oil-producing scrap residues may of course also be used.

Use of hybrid materials as fertilizer absorbers and/or as bedding material in animal husbandry may also be desirable. A combination of a fertilizer-free product with sawdust or wood shavings is also possible; this can then be dried and used as “animal bedding” in animal husbandry, especially for cattle. It is also interesting to finish the crumb subsequently with extremely find-grained synthetic polymer particles, often forming a dust, the use of which in pure form is normally problematical. Due to the adhesive effect of the fresh crumbs of the inventive product, woven or nonwoven fabrics can also be finished to be free-flowing and to be used wherever water-absorbent products in bound or secured form are desired. These include hanging gardens, inserts for shipment of goods and coffins (caskets).

If these crumb-containing woven and nonwoven fabrics are additionally furnished with floatable natural materials and synthetic plastics, they may also be used in moist areas such as plant cultivation, rice cultivation or for insect control with an appropriate finish.

Preferred applications of the hybrid material may also included the hygiene area, the cosmetics and wellness areas, where the hybrid material may be used as a component of fango [seaweed mud] packs, mud baths or mineral packs such as mineral facial or body masks.

Because of its high specific gravity and its water uptake and swelling capacity, the hybrid material may also be used in sealing applications, e.g., as an additive in systems for sealing boreholes, e.g., in petroleum drilling, as a component in sandbags for dyke repair or elevation, as a cable protective to prevent the destructive penetration of marine water into the cable or as a filler compound for elastic tubing, to be able to achieve an effective seal with respect to groundwater and rainwater at the passages in walls required for pipelines and cable installations.

It can be seen from this that the inventive products are at the same time synergistic carrier materials for a wide variety of solid and liquid products owing to their extraordinary properties and pocket structure. They may thus be used not only for water storage and as a source of nutrients but also as a depot material for an environmentally friendly means of introducing fungicides, herbicides, pesticides, etc.

The present invention is described below by the following examples which are not intended to restrict the scope of this invention in any way.

### Example 1

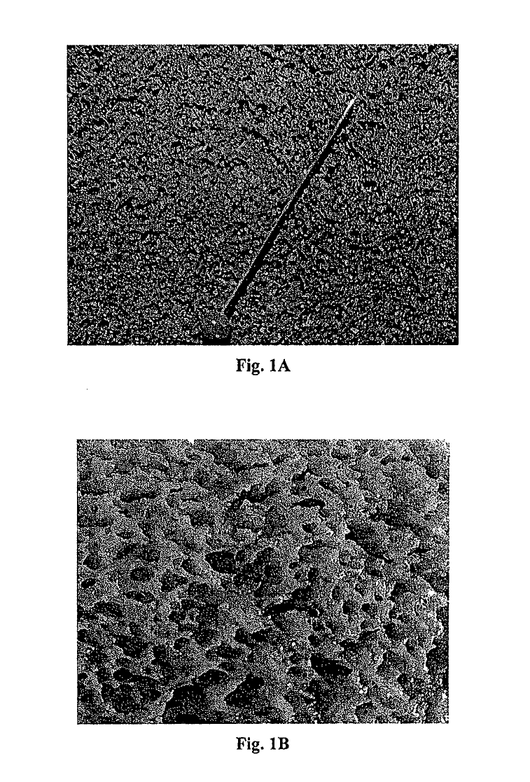

In a glass beaker, 180 g deionized water was added first and mixed with 150 g acrylic acid at room temperature. Then while stirring, 7 g urea was added and dissolved therein. The pH was about 1.6. Next, 0.02 g Wako V50 and 0.4 g butanediol diacrylate was added as the crosslinking agent. Then 460 g inorganic solids (mixture of powdered lava rock 200 g (Eifelgold from the company Lavaunion in Germany, <0.2 mm average grain size), 60 g bentonite (Agromont Calif. from S&B Minerals, <0.065 mm average grain size) and 200 g sand (from Quarzwerke Baums, L60, 0.2 mm average grain size)) were added while stirring and the slurry was homogenized. The acrylic acid was partially neutralized by adding 75 g KOH. Then the polymerization reaction was initiated by adding 0.15 g potassium disulfite, 0.9 g sodium peroxodisulfate and 0.45 g ascorbic acid (dissolved in water). In the course of the exothermic polymerization reaction, water vapor and carbon dioxide gas were released. An elastic spongy product having closed pores was formed at an average reaction temperature of 105° C. with an increase in volume to twice the initial volume of the reaction mixture. Approximately 4% of the water used was evaporated. Then the product was pulverized by means of a slowly rotating cutting tool. The resulting hybrid material had a maximum swellability (24 hours in deionized water) amounting to almost 30 times its inherent weight and had a Shore hardness of approximately 15 in the condition of being wet from production (water content approximately 35 wt %). FIG. 1A shows the spongy structure of the resulting dry material, using a needle for size comparison. FIG. 1B shows the same material in a water-saturated swollen state.

### Example 2

Using the same material as that described in Example 1, another polymerization batch was prepared, but using 260 g deionized water. The pH was about 1.6. In the course of the exothermic polymerization reaction, water vapor (approximately 2% water was evaporated) 23- and carbon dioxide gas were released at an average reaction temperature of 80° C., so the volume of the batch was increased by approximately 50%. The resulting elastic spongy product having closed pores was gently pulverized by means of a slowly rotating cutting tool. The resulting hybrid material had a maximum swellability (24 hours in deionized water) amounting to approximately 30 times its inherent weight and had a Shore hardness of approximately 20 in the condition in which it was moist from production (water content approximately 35 wt %).

### Example 3

A polymerization batch as described in Example 1 was prepared using the same materials in the amounts stated there. During the exothermic polymerization reaction, the reaction vessel was cooled in a water bath so that the average reaction temperature was kept at approximately 65° C. The volume expansion amounted to approximately 15%. The product was pulverized as described in Example 1. The resulting hybrid material had a maximum swellability (24 hours in deionized water) of approximately 25 times its inherent weight and a Shore hardness of approximately 28 in the condition of being moist from production (water content approximately 35 wt %).

### Example 4

### Comparative Example

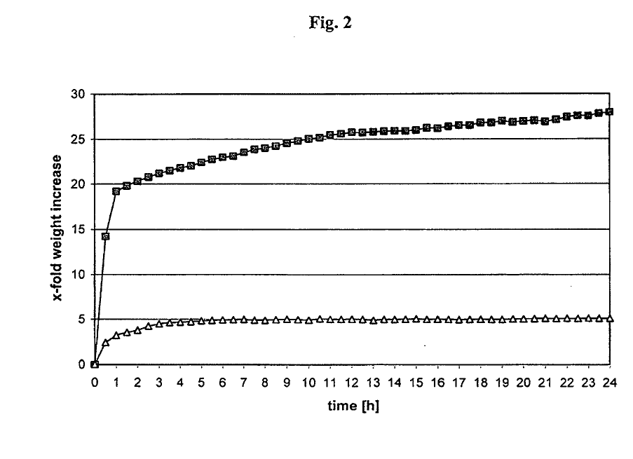

100.0 g water, 560 g potassium hydroxide solution (50%) were combined with 100.0 g acrylic acid and 40.0 g aqueous butanediol diacrylate solution (0.8 wt %), 40.0 g bentonite and 140.0 g quartz sand plus 120 g powdered lava rock (Eifel lava) in a finely ground form at a basic pH, stirred well and polymerization was initiated by adding 20.0 mL of a 1.0 wt % sodium peroxodisulfate solution, 10 mL of a 0.2 wt % ascorbic acid solution and 10 mL of a 1.25 wt % potassium disulfite solution. After about 1 minute, during which the mixture was stirred further and well, the start of polymerization could be detected on the basis of the heat release, forming microbubbles at the surface. After about 3 minutes, the mixture had become so intrinsically viscous that no sedimentation of solids was possible and the stirring was stopped. The polymer product then underwent an increase in volume in the next few minutes due to bubbling of carbon dioxide. The polymer product could easily be removed from the vessel and was pulverized by means of a cutter mill and dried by circulating air drying. FIG. 2 shows the swelling behavior of the material according to example 4 (bottom curve) in comparison with the hybrid material according to Example 1 (top curve) as a result of the hybrid material coming in contact with deionized water. The samples that were used were taken from the water after certain periods of time, placed on a screen where they were allowed to drip and then weighed. It can be seen clearly that the material according to Example 1 initially took up the water much more rapidly and had absorbed more than 20 times its own weight in water after about two hours.

### Example 5

This example shows a comparison of the development of biomass by grass in a substrate containing 1 wt % of the hybrid material from Example 1 in pure sand as the substrate. Plant containers with a diameter of 8 cm with fine sand from Haver & Boecker with the code name L 60 or fine sand mixed with 1 wt % of the hybrid material according to Example 1 and then a grass seed mixture RSM 3.1 (50% Lolium perenne, 50% Poa pratensis) was sown. For reproducibility, each test was repeated four times. Conditions: 25° C. constant, 10 kLux with a lighting time of 12 hours.

Water supply:
3 mm/d, 3-day rhythm corresponding to 57 mL every three days
1.5 mm/d, 6-day rhythm corresponding to 57 mL every six days

### Variants:

Variant 0-3/57 I-IV=pure sand, 57 mL H₂O every three days
Variant 1-3/57 I-IV=1% material from Example 1, 57 mL H₂O every three days
Variant 0-6/57 I-IV=pure sand, 57 mL H₂O every six days
Variant 1-6/57 I-IV=1% material from Example 1, 57 mL H₂O/six days

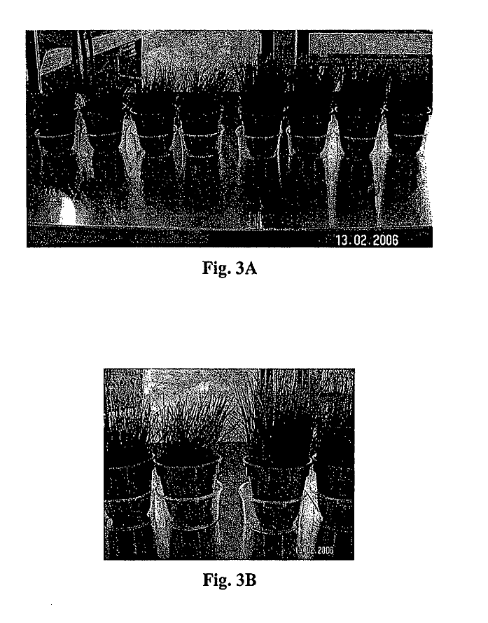

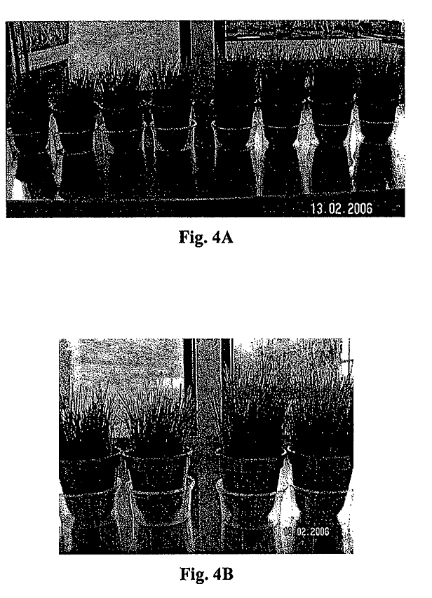

Shortly after emergence of the grass was observed, it was found that the grass to which the swellable hybrid material had been added was developing significantly more than the grass without. FIG. 3 shows the different heights of growth in the comparison of variant 0-3 without the hybrid material (four pots on the left) with variant 1-3 with hybrid material (four pots on the right), i.e., with watering of 57 mL every three days, with FIG. 3B showing an enlargement of the detail of the photograph from FIG. 3A. FIG. 4 shows the different heights of growth of the comparison of variant 0-6 without hybrid material (four pots on the left) with variant 1-6 with hybrid material (four pots on the right), i.e., with watering in the amount of 57 mL every six days, with FIG. 4B representing an enlargement of a detail of the photograph from FIG. 4A.

At three points in times, the heights of growth of the grass were measured. At all points in time, the grass height obtained with 1% water-swellable hybrid material from Example 1 was significantly higher than that of the untreated variant, namely by approximately 18% to 27% in each case. The differences were apparent with a watering schedule of 1.5 mm/d as well as that of 3 mm/d. The results show that the growth of plants can be increased by more than 20% while increasing the dry solids yield of the grass and significantly improving the water efficiency with a combination of reduced water usage and addition of the novel water-swellable hybrid material from Example 1.

### Example 6

The granules according to Example 4 were homogeneously blended with 0.1 wt % of the fungicide Parmetol® DF12 and then maximally impregnated with water. The moist granules were stored exposed to air at room temperature for 12 months and still kept moist. There was no colonization with microorganisms.

The present invention is now defined in greater detail on the basis of the accompanying claims which are fundamentally not to be interpreted in a restrictive sense.

1. A water-swellable hybrid material comprising a crosslinked polymer matrix and inorganic solid particles bound therein, wherein the hybrid material has a time-dependent swelling behavior that corresponds to a water uptake of at least 7.5 times the inherent weight of the hybrid material within one hour, and said hybrid material has been produced by providing acid-group-containing monomers of the polymer matrix first and then adding the mineral materials.

2. The hybrid material of claim 1, wherein the water uptake corresponds to at least 10 times the inherent weight of the hybrid material within the first hour.

3. The hybrid material of claim 1, wherein the inorganic solid particles include at least one material selected from quartz sand, clay, shale, sedimentary rocks, meteorite rocks, eruptive rocks, graywacke, gneiss, trass, basalt, dolomite, magnesite, bentonite, pyrogenic silica or feldspar.

4. The hybrid material of claim 1 wherein the polymer matrix includes at least one homopolymer and/or copolymer of ethylenically unsaturated components.

5. The hybrid material of claim 1, further comprising at least one water soluble additive, water-swellable additive, and/or an additive dissolved in water, selected from alkalisilicate, potassium waterglass, sodium waterglass, potassium hydroxide, sodium hydroxide, silica, alkaliphosphate, akalinitrate, alkaline earth hydrogen phosphate, phosphoric acid, boric acid, coloring agents, flavoring agents, fertilizers, urea, uric acid, guanidine, glycol, glycerol, polyethylene glycol or starch.

6. The hybrid material of any one of according to claim 1 further comprising at least one organic additive selected from the group consisting of microorganisms, bacteria, fungi, yeast, fungicides, pesticides, herbicides, cellulose, starch derivatives, plastics or polysaccharides; wood, straw, peat, recycled paper, chromium free leather and recycled granules, plastic granules, fibers and non-wovens.

7. A method of manufacturing a water-swellable hybrid material comprising the steps:
a) providing a reaction mixture comprising at least one polymerizable component and at least one suitable solvent, the pH of the reaction mixture being less than 7;
b) mixing inorganic solid particles into the reaction mixture;
c) adding at least one crosslinking agent;
d) initiating a polymerization reaction; and
e) controlling the polymerization reaction so that a spongy, water-swellable hybrid material comprising a crosslinked polymer matrix with inorganic solid particles bound therein is obtained, accompanied by an increase in volume in relation to the volume of the reaction mixture.

8. The method of claim 7, further comprising adding organic solid particles in step b).

9. The method of claim 7 wherein controlling the polymerization reaction comprises controlling the reaction heat.

10. The method of claim 9, wherein the reaction heat is controlled such that from about 0.1 to 30 wt.-% of the at least one solvent is vaporized.

11. The method of claim 9 wherein the reaction heat is controlled via the quantity ratio of the at least one polymerizable component to the at least one suitable solvent, or the volume of the solvent.

12. The method of claim 11, wherein the quantity ratio of the at least one polymerizable component to the at least one suitable solvent is between about 1:1 to 1:5.

13. The method of claim 9, wherein the reaction heat is controlled by cooling of the reaction mixture.

14. The method of claim 7, wherein the increase in volume relative to the volume of the reaction mixture before initiating the polymerization reaction is at least 10%.

15. The method of claim 7, wherein the increase in volume is at least partially effected by a suitable amount of at least one gas evolving substance in the reaction mixture.

16. The method of claim 7, wherein the polymerization step has an average reaction temperature of about 50° C. to 130° C. and an initial temperature of about 4° C. to about 40° C.

17. The method of claim 7, wherein the solvent comprises a protic-polar solvent.

18. The method of claim 7, wherein the pH value of step a) is below pH 6.5.

19. The method of claim 7, wherein the polymerizable component is a water-soluble ethylenically unsaturated monomer selected from the group consisting of acrylic acid, methacrylic acid, ethacrylic acid, sorbic acid, maleic acid, fumaric acid, itaconic acid, vinylsulfonic acid, methacrylaminoalkylsulfonic acid, vinylphosphonic acid and vinylbenzenephosphonic acid.

20. The method of any one of further comprising at least one water-soluble, ethylenically unsaturated comonomer selected from the group consisting of acrylamide, methacrylamide, N-alkylacrylamide, N-alkylmethacrylamide, N-dialkylaminoacrylamide, N-dialkylaminomethacrylamide, N-methylolacrylamide, N-methylolmethacrylamide, N-vinylamide, N-vinylformamide, N-vinylacetamide, N-vinyl-n-methylacetamide, N-vinyl-n-methylacetamide, N-vinyl-n-formamide, vinylpyrrolidone, hydroxyethyleneacrylate, hydroxyethylmethacrylate, acrylic acid esters and methacrylic acid esters.

21. The method of claim 7, wherein the crosslinking agent is selected from compounds having at least two ethylenically unsaturated groups, or at least one ethylenically unsaturated group and at least one functional group reactive with acid groups.

22. The method of claim 21, wherein the crosslinking agent is selected from the group consisting of methylenbisacrylamide, mono-, di- and polyesters of acrylic acid, methacrylic acid, itaconic acid, maleic acid, esters of these acids with allyl alcohol or its alkoxylated homologs, polyvalent alcohols, butanediol, hexanediol, polyethylene glycol, trimethylolpropane, pentaerythritol, glycerol, polyglycerol as well as the alkoxylated homologs of these polyvalent alcohols, dihydroxyalkylmonoester, butanediol diacrylate; allylacrylamide, triallyl citrate, trimonoallyl, polyethylene glycol ether citrate, N-diallyl-acrylamide, diallyl phthalate, triallyl citrate, tri-monoallyl-polyethylene glycol ether citrate, allyl ethers of diols and polyols and their ethoxylates, polyallyl ethers of glycerol, trimethylol propane, pentaerythritol and the ethoxylates thereof, tetra-allyloxyethane and polyglycidylallyl ether, ethylene glycol diglycidyl ether, glycerol glycidyl ether; diamines and their salts with at least two ethylenically unsaturated substituents; diamine or triallylamine, or tetra-allylammonium chloride.

23. The method of claim 7, wherein the polymerization is initiated by at least one suitable redox system or by photocatalysis in the presence of suitable sensitizers or combinations thereof.

24. The method of claim 7, further comprising thermally or chemically treating the hybrid material to remove residual monomer, to post-crosslinking, partial hydrolysis and/or for drying by heating in a convection oven, with superheated steam at temperatures of about 100 to about 150° C., or by injecting heated gases under pressure.

25. (canceled)

26. The hybrid material of claim 1 having a residual moisture content of at least about 0.1 wt.-% by total weight of the moist material.

27. The hybrid material of claim 1 having a Shore A Hardness (DIN 53505) of at least about 25 after 12 hours of drying of the hybrid material at about 40° C.

28. The hybrid material of claim 27, wherein the Shore A Hardness (DIN 53505) is at least about 1 in a saturated state after storing the material for 24 hours in deionized water.

29. (canceled)

30. A soil additive comprising a hybrid material a water-swellable hybrid material comprising a crosslinked polymer matrix and inorganic solid particles bound therein, wherein the hybrid material has a time-dependent swelling behavior that corresponds to a water uptake of at least 7.5 times the inherent weight of the hybrid material within one hour, and at least one substance selected from the group consisting of soil, humus, sand, peat and the like.

31. A method for storing and delivering water and/or active agents comprising adding water and/or active ingredients to a water-swellable hybrid material comprising a crosslinked polymer matrix and inorganic solid particles bound therein, wherein the hybrid material has a time-dependent swelling behavior that corresponds to a water uptake of at least 7.5 times the inherent weight of the hybrid material within one hour, and applying the hybrid material to landscaping soil to absorb or release water, fertilizers, pesticides, fungicides, microorganisms and/or in combination with seeds.
